(19) United States
(12) Patent Application Publication
(10) Pub. No.: US 2022/0361800 A1
(43) Pub. Date: Nov. 17, 2022

(54) ELECTROCARDIOGRAM SIGNAL PROCESSING METHOD AND APPARATUS

(71) Applicant: ATSENS CO., LTD., Gyeonggi-do (KR)

(72) Inventors: Kab Mun CHA, Seoul (KR); Jong Ook JEONG, Gyeonggi-do (KR)

(73) Assignee: ATSENS CO., LTD., Gyeonggi-do (KR)

(21) Appl. No.: 17/724,862

(22) Filed: Apr. 20, 2022

(30) Foreign Application Priority Data
May 4, 2021 (KR) 10-2021-0058112

(51) Int. Cl. A61B 5/361; A61B 5/347

(52) U.S. Cl. CPC A61B 5/361; A61B 5/347

(57) ABSTRACT

An electrocardiogram signal processing apparatus includes a controller and a communication unit transmitting an electrocardiogram signal. The controller is configured to (i) extract points satisfying a determined standard from the electrocardiogram signal and convert the extracted points into a two-dimensional graph expressed as frequencies for a plurality of class sections, (ii) generate a cumulative graph in which an order of the plurality of class sections is rearranged in an order of magnitude of the frequencies, and (iii) calculate a Gini index based on the cumulative graph and determine whether the electrocardiogram signal is an abnormal signal by using the Gini index.

### CROSS-REFERENCE TO RELATED APPLICATION

This application is based on and claims priority under 35 U.S.C. § 119 to Korean Patent Application No. 10-2021-0058112, filed on May 4, 2021, in the Korean Intellectual Property Office, the disclosure of which is incorporated by reference herein in its entirety.

### BACKGROUND

### 1. Field

One or more embodiments relate to an electrocardiogram signal processing method and apparatus, and more particularly to, an electrocardiogram signal processing method and apparatus for determining whether an electrocardiogram signal is an abnormal signal by minimizing the influence of regular fluctuations.

### 2. Description of the Related Art

Atrial fibrillation (AF) is a typical arrhythmia symptom that causes irregular heartbeat, which may eventually lead to blood clots and stroke to cause death. Currently, 2.2 millions or more of people in the United States have atrial fibrillation symptoms, and it is expected that the number will be doubled over the next 20 years.

Various algorithms have been developed to detect an irregular heart rhythm, such as AF or atrial flutter (AFL).

The detection of AF based on the absence of a P wave has a problem in that it is difficult to determine a position of a reference point for detecting the P wave due to human movement and other noise artifacts.

An algorithm for detecting AF based on the irregularity of an R-R interval (RRI) may classify normal sinus rhythm (NSR) and AF with excellent performance. However, this algorithm is not suitable for classifying an AF state and a non-AR state when there is ‘regular fluctuation’ such as an atrial premature beat (APB). Such “regular fluctuation” is easily found even in the absence of heart disease, and very rarely cause symptoms. Accordingly, it is necessary to distinguish the regular fluctuation from an AF state.

Korean Patent Publication Registration No. 10-1912090 discloses a technique for generating an AF prediction model and a technique for predicting AR. The technique for predicting AF extracts important feature points of a T wave by analyzing electrocardiogram data of an object to be measured, the electrocardiogram data being collected in real time, and retrieves an AF aspect corresponding to the important feature points of the extracted T wave of the object to be measured from a pre-stored AF prediction model to predict the possibility of AF.

However, similarly to the P wave, the T wave has a small signal value and is vulnerable to noise caused by the movement of a human body.

Accordingly, there is a need for a new algorithm capable of more accurately determining whether an electrocardiogram signal includes an abnormal signal by minimizing the effect of regular fluctuation.

### SUMMARY

One or more embodiments include an electrocardiogram signal processing method and apparatus for determining whether an electrocardiogram signal is an abnormal signal by minimizing the influence of regular fluctuations.

Additional aspects will be set forth in part in the description which follows and, in part, will be apparent from the description, or may be learned by practice of the presented embodiments of the disclosure.

According to one or more embodiments, an electrocardiogram signal processing method includes an operation in which an electrocardiogram signal processing apparatus receives an electrocardiogram signal, an operation in which the electrocardiogram signal processing apparatus extract values satisfying a determined standard from the electrocardiogram signal and convert the extracted values into a two-dimensional graph representing frequencies of a plurality of class sections, an operation in which the electrocardiogram signal processing apparatus generates a cumulative graph in which an order of the plurality of class sections is rearranged in an order of magnitude of the frequencies, an operation in which the electrocardiogram signal processing apparatus calculates a Gini index based on the cumulative graph, and an operation in which the electrocardiogram signal processing apparatus determines whether the electrocardiogram signal is an abnormal signal by using the Gini index.

In an embodiment, the operation in which the electrocardiogram signal processing apparatus determines whether the electrocardiogram signal is an abnormal signal may include determining the electrocardiogram signal is an abnormal signal when the Gini index is equal to or less than a preset reference value.

In an embodiment, the reference value may be determined as a result of learning through a plurality of electrocardiogram signals obtained by a plurality of users.

In an embodiment, the abnormal signal may include information on whether the electrocardiogram signal from a user has an arrhythmia or an atrial fibrillation.

In an embodiment, the two-dimensional graph may be obtained by dividing a class section with respect to values or time intervals of the electrocardiogram signal.

In an embodiment, the class sections may be divided by changeable standards.

In an embodiment, the values satisfying the determined standard may be time values between reference points of a heartbeat cycle, and the two-dimensional graph may be obtained by dividing the class section with respect to time, and the two-dimensional graph may represent a time interval between the reference points of the heartbeat cycle as frequencies for the plurality of class sections.

According to one or more embodiments, an electrocardiogram signal processing apparatus includes a controller and a communication unit transmitting an electrocardiogram signal, wherein the controller is configured to extract points satisfying a determined standard from the electrocardiogram signal and to convert the extracted points into a two-dimensional graph expressed as frequencies for a plurality of class sections, to generate a cumulative graph in which an order of the plurality of class sections is rearranged in an order of magnitude of the frequencies, to calculate a Gini index based on the cumulative graph and determine whether the electrocardiogram signal is an abnormal signal by using the Gini index.

In an embodiment, the controller may be is further configured to determine the electrocardiogram signal is an abnormal signal when the Gini index is equal to or less than a preset reference value.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electrocardiogram signal processing apparatus according to an embodiment;

FIG. 2 is a block diagram of a controller shown in FIG. 1;

FIG. 3 is a block diagram of an electrocardiogram signal processing apparatus according to an embodiment;

FIG. 4 is a flowchart illustrating an electrocardiogram signal processing method according to an embodiment;

FIG. 5 is a flowchart illustrating a method of determining whether an electrocardiogram signal is an abnormal signal, according to an embodiment;

FIG. 6 is a diagram for explaining an electrocardiogram signal processing network according to an embodiment;

FIG. 7 is a block diagram of the electrocardiogram signal processing network shown in FIG. 6;

FIG. 8 is a flowchart illustrating a method of determining a reference value of a Gini index;

FIG. 9A is an exemplary diagram of a graph of a potential difference with time generated according to an embodiment;

FIG. 9B is an exemplary diagram of a two-dimensional graph generated according to an embodiment;

FIG. 9C is an exemplary diagram of a graph rearranged in the order of magnitude of frequencies generated according to an embodiment;

FIG. 9D is an exemplary diagram of a cumulative graph for frequencies generated according to an embodiment; and

FIG. 9E is a diagram illustrating an operation of calculating a Gini index according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Various modifications and variations may be applied to the present disclosure, and particular embodiments of the present disclosure will be described in detail hereinbelow with reference to the drawings. The present disclosure is not limited to the embodiments set forth herein, but all changes, equivalents, and substitutes that do not depart from the spirit and technical scope are encompassed in the present disclosure.

The present disclosure may be described in terms of functional block components and various processing steps. Such functional blocks may be realized by any number of hardware and/or software components configured to perform the specified functions. For example, the present disclosure may employ various integrated circuit (IC) components, e.g., memory elements, processing elements, logic elements, look-up tables, and the like, which may carry out a variety of functions under the control of one or more microprocessors or other control devices.

Similarly, where the elements are implemented using software programming or software elements, the inventive concept may be implemented with any programming or scripting language such as C, C++, Java, assembler language, or the like, with the various algorithms being implemented with any combination of data structures, objects, processes, routines or other programming elements.

Furthermore, the present disclosure could employ any number of conventional techniques for electronics configuration, signal processing and/or control, data processing and the like. The terms “unit,” “element,” “means,” and “configuration” are used broadly and are not limited to mechanical or physical embodiments. The terms can include software routines in conjunction with processors, etc.

It will be understood that although the terms “first,” “second,” etc. may be used herein to describe various elements, components, areas, layers and/or steps, these elements, components, areas, layers and/or steps should not be limited by these terms.

The present disclosure will now be described more fully with reference to the accompanying drawings, in which exemplary embodiments of the present disclosure are shown. Like reference numerals in the drawings denote like elements, and thus their description will be omitted.

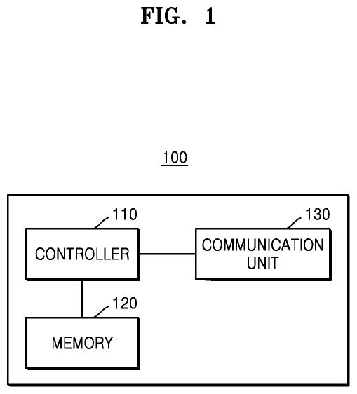

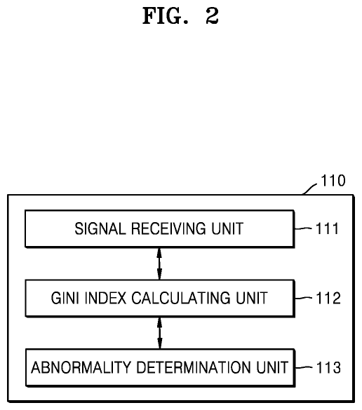

FIGS. 1 and 2 are block diagrams of an electrocardiogram signal processing apparatus 100 according to an embodiment. FIG. 2 illustrates a block diagram of a controller 110 shown in FIG. 1.

Referring to FIG. 1 the electrocardiogram signal processing apparatus 100 according to an embodiment of the present disclosure may be an apparatus that receives an electrocardiogram signal from an external electrocardiogram signal measuring device or a device storing electrocardiogram signal data to determine whether the electrocardiogram signal is a normal or abnormal signal. The electrocardiogram signal processing apparatus 100 may be a computing device including a controller 110, a memory 120, and a communication unit 130. Such a computing device may achieve desired system performance by using a combination of computer hardware (e.g., a device that may include, for example, a computer processor, a memory, a storage, an input device, an output device, and other components of a computing device in the related art) and computer software (i.e., instructions that cause a computing device to function in a particular way). As an embodiment, the electrocardiogram signal processing apparatus 100 may be a user terminal device such as a mobile phone, a tablet, or a personal computer. As another embodiment, the electrocardiogram signal processing apparatus 100 may also be a network server of an institution that provides medical services, such as a hospital, a research institute, or a company.

The memory 120 may store a program for processing or controlling the controller 110 and the communication unit 130 and various pieces of data for the overall operation of the electrocardiogram signal processing apparatus 100. The memory 120 may store a plurality of application programs driven by the electrocardiogram signal processing apparatus 100, data for an operation of the electrocardiogram signal processing apparatus 100, and instructions. At least some of the plurality of application programs, data, and instructions may be downloaded from an external server through the communication unit 130 or may be temporarily shared. In addition, at least some of the plurality of application programs, data, and instructions may exist on the electrocardiogram signal processing apparatus 100 from the time of release of the electrocardiogram signal processing apparatus 100 for a basic function of the electrocardiogram signal processing apparatus 100.

For example, the memory 120 may be implemented as an internal memory such as read-only memory (ROM), random-access memory (RAM), or the like in the controller 110, or may be implemented as a memory separate from the controller 110.

The communication unit 130 may be configured to communicate with various types of external devices according to various types of communication methods. The communication unit 130 may receive electrocardiogram signal data from an electrocardiogram signal measuring device or a server of a network institution that provides medical services, or the like. Also, the communication unit 130 may receive or update an application or data required for driving the electrocardiogram signal processing apparatus 100. In addition, the communication unit 130 may transmit, to a computing device, a smartphone, a server, or the like, a Gini index calculated by the electrocardiogram signal processing apparatus 100 and/or a determination result of whether an electrocardiogram signal is an abnormal signal.

As an embodiment, the communication unit 130 may include a device for wireless communication, such as a Wi-Fi chip, a Bluetooth chip, a wireless communication chip, or a near-field communication (NFC) chip. The wireless communication chip refers to a chip that performs communication according to various communication standards such as the Institute of Electrical and Electronics Engineers (IEEE), 3rd Generation (3G), 3rd Generation Partnership Project (3GPP), Long Term Evolution (LTE), or the like. The NFC chip refers to a chip operating in an NFC method using a 13.56 MHz band among various radio frequency-identification (RF-ID) frequency bands such as 135 kHz, 13.56 MHz, 433 MHz, 860 MHz to 960 MHz, 2.45 GHz, or the like. As another embodiment, the communication unit 130 may include a device for wired communication using a communication line to exchange data with an external device. The device for wired communication may include a network interface controller or the like.

The controller 110 is configured to generally control the electrocardiogram signal processing apparatus 100. In particular, the controller 110 controls the overall operation of the electrocardiogram signal processing apparatus 100 by using various types of programs stored in the memory 120 of the electrocardiogram signal processing apparatus 100. The controller 110 may include at least one central processing unit (CPU). The controller 110 may be implemented as a digital signal processor (DSP) processing a digital signal, a microprocessor, or a time controller (TCON). However, the present disclosure is not limited thereto, and the controller 110 may include one or more of a CPU, a micro controller unit (MCU), a controller, an application processor (AP), a communication processor (CP), and an Advanced RISC Machines (ARM) processor, or may be defined by a corresponding term. In addition, the controller 110 may be implemented as a system on chip (SoC) having a built-in processing algorithm, large scale integration (LSI), or may be implemented in a form of a field programmable gate array (FPGA).

Hereinafter, the controller 110 is described in more detail below with reference to FIG. 2. Here, each function unit may be implemented with various numbers of hardware and/or software components that perform particular functions.

The controller 110 may include a signal receiving unit 111 receiving an electrocardiogram signal, a Gini index calculating unit 112 calculating a Gini index from the electrocardiogram signal, and an abnormality determination unit 113 determining whether the electrocardiogram signal is an abnormal signal based on the calculated Gini index.

The signal receiving unit 111 receives a signal measured by an electrocardiogram signal measuring sensor through the communication unit 130. Here, the electrocardiogram signal is a signal for measuring an electrical activity of a heart by attaching at least one electrode to a part of a body. As an embodiment, in the electrocardiogram signal, a potential value of a part of a body to which an electrode is attached, a difference in potential values measured by a plurality of electrodes, or a value calculated based on the potential value and the difference in potential values may be recorded as a continuous waveform over time. As an embodiment, in the electrocardiogram signal, a current value of a part of a body to which an electrode is attached, current values measured by a plurality of electrodes, or a value calculated based on the current values may be recorded as a continuous waveform over time. As another embodiment, in the electrocardiogram signal, a value calculated based on the above-mentioned potential value and current value may be recorded as a continuous waveform over time.

The Gini index calculating unit 112 calculates a Gini index based on an electrocardiogram signal.

The Gini index calculating unit 112 may firstly extract values satisfying a determined standard from an electrocardiogram signal, and calculate a Gini index indicating a degree of distribution balance of the extracted values. Here, the determined standard may be based on time or a signal value. For example, the values satisfying the determined standard may be R-R interval length values of an electrocardiogram signal, and time values of particular waveform the electrocardiogram signal. The Gini index may be calculated according to a degree of distribution balance of values extracted based on various standards.

The Gini index calculating unit 112 may set windows of a certain size for entire or a portion of an electrocardiogram signal, and calculate a Gini index by representing electrocardiogram signal values in each window as a degree of distribution balance between the windows. Here, the window may be defined by a time section, a value section, or the like, for example, may be defined as a window with an interval of 10 seconds, a window of 0.1 mV, or the like. A window defined by a time section may move along a time axis in an electrocardiogram signal. A window defined by a section of a signal value may move along an axis of a signal value in an electrocardiogram signal. A window may be slid according to a preset interval. The number of windows according to a preset size, for example, may be slid at intervals of 30 electrocardiogram signal measurement points.

As an embodiment, the values satisfying the determined standard may be time interval values between reference points in an electrocardiogram signal that are repeatedly generated every heartbeat cycle. Here, the reference points may refer to points corresponding to a particular wave periodically generated in an electrocardiogram signal. For example, the reference point may be one of periodically generated points such as a P wave, an R wave, or the like. When a first reference point, a second reference point, and a third reference point are successively generated for each heartbeat cycle in an electrocardiogram signal, a first time interval value between the first reference point and the second reference point and a second time interval value between the second reference point and the third reference point may be values satisfying a determined standard.

As another embodiment, values satisfying a determined standard may be electrocardiogram signal measurement values of reference points of a heartbeat cycle. An electrocardiogram signal measurement value may be a potential value measured by at least one electrode, a current value measured by at least one electrode, or a value calculated based on the potential value and the current value. As an embodiment, the electrocardiogram signal measurement value may be a potential value of a part of a body to which one electrode is attached based on a particular point on the body. As another embodiment, an electrocardiogram signal measurement value may be a difference between potential values of a part of a body to which each of two electrodes is attached.

Reference points of a heartbeat cycle may be a point having a maximum value within a preset time section, a point having a minimum value within a preset time section, a peak point of a P wave, each peak point of a QRS wave, or a peak point of a T wave, but is not limited thereto. The reference points of the heartbeat cycle may be selected by considering characteristics of an electrocardiogram signal measuring device and a measuring environment.

The Gini index calculating unit 112 may convert extracted values into a two-dimensional graph representing frequencies for a plurality of class sections.

The two-dimensional graph may be a two-dimensional graph of a plurality of class sections and the frequencies of each class section. The plurality of class sections may be defined according to a determined standard used for extracting values. When values are extracted based on the amplitude of measured values, the class sections may be defined as the amplitude section of the measured values. A plurality of class sections may be defined by dividing a difference value between maximum and minimum values of the measured values into a certain size. When the values are extracted based on the length of the time interval between the reference points, the class sections may be defined as a section of a length of time interval between the reference points. A plurality of class sections may be defined by dividing a difference value between maximum value and minimum values of a time interval length between reference points at regular intervals.

A standard that is used to divide the class sections may be changed according to the measurement precision of an electrocardiogram signal measuring device, a measurement environment, and a purpose of the measurement.

The Gini index calculating unit 112 may convert extracted values into a two-dimensional graph by representing the extracted values as frequencies for the corresponding class section. The Gini index calculating unit 112 may distribute the extracted values to each class section including each value. The Gini index calculating unit 112 may calculate a number of distribution frequencies of each class section as a frequency. In a two-dimensional graph, the frequency may be used as one axis (e.g., a y axis), and a class value (an intermediate value between an upper limit value and a lower limit value of a class section) may be used as the other axis (e.g., an x axis).

The Gini index calculating unit 112 may generate a cumulative graph in which an order of class sections in the two-dimensional graph is rearranged according to an order of magnitude of the frequencies. First, the Gini index calculating unit 112 may rearrange the order of class sections in the two-dimensional graph according to the order of magnitude of the frequencies. For example, when the frequency of the first class section is 10, the frequency of the second class section is 3, and the frequency of the third class section is 8, we may rearrange in an ascending order according to the order of magnitude of the frequencies to be the second class section, the third class section, and the first class section.

The Gini index calculating unit 112 may convert the rearranged graph into a graph regarding a relative frequency (a frequency value of each class section/a total frequency value), and convert the graph into a cumulative graph. Here, the cumulative graph may be a Lorentz curve having a maximum value of 1. From the Lorentz curve, a degree of equality or inequality in the number of frequencies for each class section of values satisfying a determined standard may be known.

Here, a Gini index is a coefficient indicating a ratio between area values of a first area between a reference line and a cumulative graph and a second area at an upper end of the reference line, with respect to the reference line connecting points where a cumulative value of a relative frequency becomes 1 from an origin. The Gini index is a value indicating the impurity of raw data and may be used as a standard for determining an abnormal electrocardiogram signal by minimizing an effect of ‘regular fluctuation’.

The abnormality determination unit 113 may determine whether an electrocardiogram signal is an abnormal signal based on a calculated Gini index. The abnormality determination unit 113 may compare the calculated Gini index with a preset threshold value, and, when the calculated Gini index is equal to or less than the threshold value, may determine that the electrocardiogram signal is an abnormal signal. Here, the threshold value may be a Gini index calculated from an abnormal electrocardiogram signal. In an embodiment, the abnormality determination unit 113 may determine whether an electrocardiogram signal is an abnormal signal in units of each window, but is not limited thereto. As an embodiment, the abnormality determination unit 113 may determine whether an abnormality is present by using a machine learning model. Here, the machine learning model may use an algorithm for binary classification, and may use decision tree learning, a perceptron, a support vector machine, or a combination thereof, but is not limited thereto.

Here, the threshold value may be determined as a result of learning through a plurality of electrocardiogram signals obtained by a plurality of users. The threshold value may be calculated by a network server of an institution that provides medical services, such as a hospital, a research institute, or a company, to be provided through the communication unit 130 of the electrocardiogram signal processing apparatus 100. Also, the threshold value may be updated by receiving a diagnosis from the electrocardiogram signal processing apparatus 100 and a doctor.

The electrocardiogram signal processing apparatus 100 according to an embodiment of the present disclosure may convert an electrocardiogram signal having a large capacity into a Gini index. In addition, it is possible to more accurately determine whether an electrocardiogram signal is an abnormal signal, independent upon a user's movement, noise during measurement, changes in a basal end of an electrocardiogram signal due to measurement errors, or an effect of regular fluctuation such as atrial contraction, by using the Gini index.

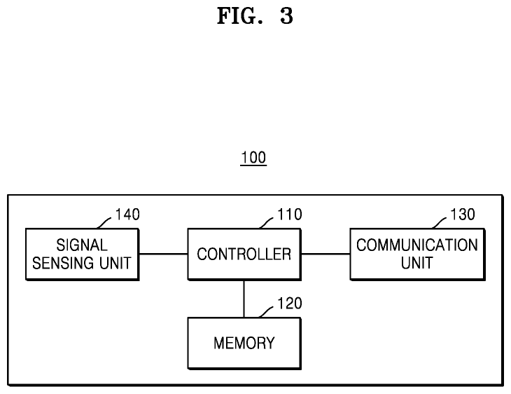

FIG. 3 is a block diagram of the electrocardiogram signal processing apparatus 100 according to an embodiment.

Referring to FIG. 3, the electrocardiogram signal processing apparatus 100 may further include a signal sensing unit 140, and may receive an electrocardiogram signal obtained by the signal sensing unit 140 to determine whether the electrocardiogram signal is an abnormal signal. As an embodiment, the electrocardiogram signal processing apparatus 100 may process the electrocardiogram signal obtained by the signal sensing unit 140 in real time to determine an abnormal section of the electrocardiogram signal.

The signal sensing unit 140 may be implemented by including at least one electrode attached to a part of a body and a sensor measuring a potential or current value of the part of the body to which the at least one electrode is attached, and may be implemented as a separate device.

As an embodiment, the electrocardiogram signal processing apparatus 100 may be manufactured in a patch type to obtain an electrocardiogram signal in the daily life of an object to be examined, but is not limited thereto. The signal sensing unit 140 may process to store an electrocardiogram signal value measured by the electrocardiogram signal processing apparatus 100 or processing data of the electrocardiogram signal value in the memory 120. The controller 110 may calculate a Gini index from an electrocardiogram signal and determine whether the electrocardiogram signal is an abnormal signal based on the Gini index. The communication unit 130 may transmit, to a user terminal and/or a network server of an institution providing medical services, data including whether an electrocardiogram signal is an abnormal signal or a Gini index calculated from the electrocardiogram signal. The communication unit 130 may be implemented to transmit data such as data whether an electrocardiogram signal is an abnormal signal, in response to a request from an external electronic device.

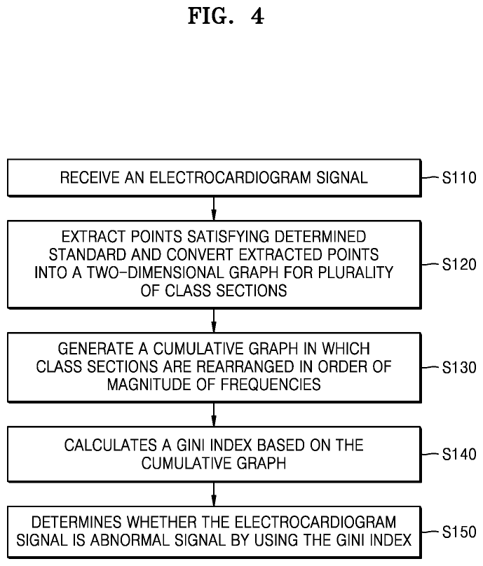

As another embodiment, the electrocardiogram signal processing apparatus 100 may be an apparatus installed in an institution that provides medical services, such as a hospital, a research institute, or a company, to examine an electrocardiogram signal of an object more precisely to be examined for a certain time period. At this time, the electrocardiogram signal processing apparatus 100 may operate as a portion of a general electrocardiogram signal measuring device or a motion-load electrocardiogram signal measuring device. FIG. 4 is a flowchart illustrating an electrocardiogram signal processing method according to an embodiment.

Referring to FIG. 4, in operation S110, an electrocardiogram signal processing apparatus may receive an electrocardiogram signal. As described above, the electrocardiogram signal processing apparatus may receive an electrocardiogram signal data from an electrocardiogram signal measuring device or a network server of an institution providing medical services. As another embodiment, the electrocardiogram signal processing apparatus may process a signal obtained by a signal sensing unit provided therein in real time to determine whether the signal is an abnormal signal.

In operation S120, the electrocardiogram signal processing apparatus may extract values satisfying a determined standard from the received electrocardiogram signal, and convert the extracted values into a two-dimensional graph representing frequencies for a plurality of class sections.

The electrocardiogram signal processing apparatus sets a window of a certain time-interval size for calculating a Gini index with respect to the firstly received electrocardiogram signal.

Values satisfying a determined standard are extracted from an electrocardiogram signal within a window. As an embodiment, the values satisfying a determined standard may be time interval values between reference points of a heartbeat cycle. As another embodiment, the values satisfying a determined standard may be electrocardiogram signal measurement values between reference points of a heartbeat cycle.

The electrocardiogram signal processing apparatus may convert the extracted values into a two-dimensional graph. Here, the two-dimensional graph may be a histogram in which class sections are divided by dividing measurement values of an electrocardiogram signal or time between reference points, and the number of frequencies of extracted values in each class section is used as a frequency.

In operation S130, the electrocardiogram signal processing apparatus may rearrange an order of class sections in the two-dimensional graph according to the order of magnitude of the frequencies, and generate a cumulative graph obtained by converting each frequency into a relative frequency. Here, the cumulative graph may be a Lorentz curve having a maximum value of 1.

In operation S140, the electrocardiogram signal processing apparatus may calculate a Gini index based on the cumulative graph. The Gini index is a coefficient that sets a reference line connecting points where a cumulative value of a relative frequency is 1 from an origin and indicates a ratio of a first area between the reference line and the cumulative graph and a second area at an upper end of the reference line.

In operation S150, the electrocardiogram signal processing apparatus may determine whether an electrocardiogram signal is an abnormal signal by using the Gini index. The electrocardiogram signal processing apparatus compares the calculated Gini index with a preset threshold value, and, when the calculated Gini index is equal to or less than the threshold value, determines that the electrocardiogram signal is an abnormal signal.

Here, the threshold value may be determined as a result of learning through a plurality of electrocardiogram signals obtained by a plurality of users. The threshold value may be a pre-stored value when the electrocardiogram signal processing apparatus is released. The threshold value may be a value updated through connection with an external network.

The electrocardiogram signal processing apparatus may determine whether an abnormal signal is present to transmit a determination result to a user terminal or a network server of an institution.

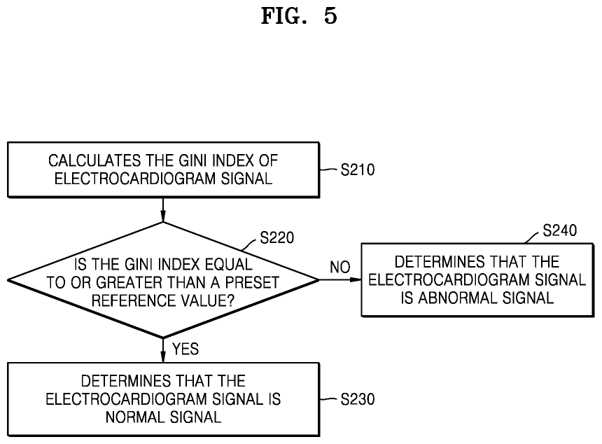

FIG. 5 is a flowchart illustrating a method of determining whether an abnormal signal is present.

Referring to FIG. 5, in operation S210, an electrocardiogram signal processing apparatus calculates a Gini index of an electrocardiogram signal. The Gini index may be calculated for each window, and may determine whether an electrocardiogram signal is a normal or abnormal signal in units of windows, but is not limited thereto. As an embodiment, the electrocardiogram signal processing apparatus may determine whether an electrocardiogram signal is a normal or abnormal signal based on a Gini index of a consecutive preset number of windows. This may correspond to whether a heart condition of an object of the electrocardiogram signal is normal or abnormal.

In operation S220, the electrocardiogram signal processing apparatus compares the Gini index with a reference value to determine whether the Gini index is equal to or greater than a preset reference value.

In operation S230 and operation S240, when the calculated Gini index is equal to or greater than the preset reference value, the electrocardiogram signal processing apparatus determines that the electrocardiogram signal is a normal signal, and when the calculated Gini index is less than the preset reference value, the electrocardiogram signal processing apparatus determines that the electrocardiogram signal is an abnormal signal.

The electrocardiogram signal processing apparatus may transmit, to a network server of an institution providing medical services, a determination result such as a result whether the electrocardiogram signal is an abnormal signal and data on an abnormal section. At this time, the determination result may be transmitted together with a value of the calculated Gini index.

The determination result of whether the electrocardiogram signal is an abnormal signal may be transmitted to a user through a user terminal. As an embodiment, when the user terminal is a mobile phone or a computing device having a monitor, the determination result of whether an electrocardiogram signal is an abnormal signal may be displayed. As another embodiment, when the electrocardiogram signal processing apparatus is an apparatus that measures an electrocardiogram signal in real time, a result of whether the electrocardiogram signal is an abnormal signal may be transmitted to a user through light-emitting diode (LED) lighting or an alarm. Here, the user may be a medical institution worker, such as a doctor or a nurse, or an object to be examined.

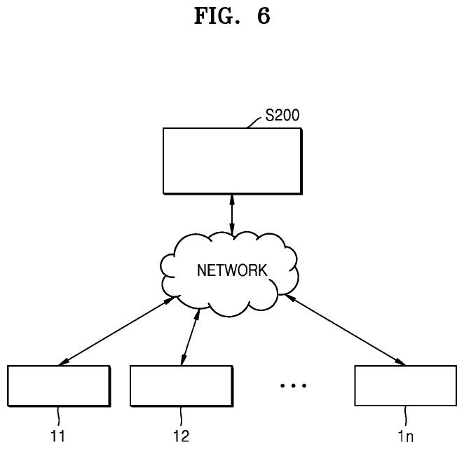

FIG. 6 is a diagram of an electrocardiogram signal processing network according to an embodiment.

Referring to FIG. 6, the electrocardiogram signal processing network may include a server 200 and at least one user terminal 11, 12, . . . , and 1n.

At this time, the at least one user terminal 11, 12, . . . , and 1n may be an electrocardiogram signal measuring device, an electrocardiogram signal processing apparatus, or a data management device of a medical service institution. The at least one user terminal 11, 12, . . . , and 1n may transmit, to the server 200, data such as raw data of a measured electrocardiogram signal, a result of determining whether the electrocardiogram signal is an abnormal signal, and/or a Gini index of the electrocardiogram signal.

The server 200 may refer to all types of servers for integrated management of the at least one user terminal 11, 12, . . . , and 1n. As an embodiment, the server 200 may store and manage electrocardiogram signal data for each user or object to be examined. As another embodiment, the server 200 may generate a signal determination model based on the electrocardiogram signal data and input an electrocardiogram signal to the signal determination model to output data for an abnormal section of the electrocardiogram signal. The server 200 may transmit the output data to the at least one user terminal 11, 12, . . . , and 1n. As a method of generating a signal determination model, a machine learning method such as unsupervised learning, supervised learning, or reinforcement learning may be used.

As another embodiment, the server 200 may calculate Gini indices from electrocardiogram signals classified as normal signals, and determine a threshold value based on the Gini indices of the normal signals. A determination model or algorithm for determining whether an electrocardiogram signal is an abnormal signal by using the threshold value determine in this way may be transmitted to the at least one user terminal 11, 12, . . . , and 1n, and may provide and update a standard for determining whether an electrocardiogram signal is an abnormal signal.

The server 200 may generate information necessary for the user based on electrocardiogram signal data and data including a result of determining whether an electrocardiogram signal is an abnormal signal. As an embodiment, the server 200 may provide information on a heart disease of objects by using information on an abnormal signal and/or a normal signal of an electrocardiogram signal obtained from a signal processing apparatus once or repeatedly.

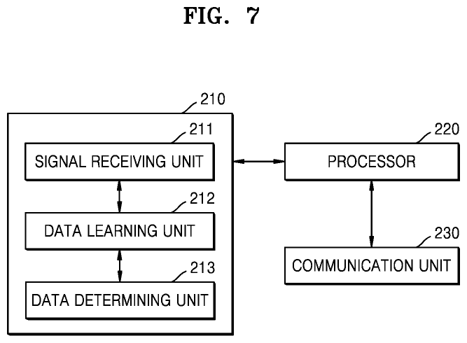

FIG. 7 is a block diagram of the server 200 shown in FIG. 6.

Referring to FIG. 7, the server 200 may include a signal determination model unit 210, a processor 220, and a communication unit 230.

The signal determination model unit 210 may generate a model for determining whether an electrocardiogram signal is an abnormal signal, evaluate the generated model, and provide a model that satisfies a certain evaluation standard. The signal determination model unit 210 may include a data receiving unit 211, a data learning unit 212, and a data determining unit 213.

The data receiving unit 211 may receive electrocardiogram signal data from the communication unit 230. The data receiving unit 211 may receive electrocardiogram signal data from a system necessary for medical services and researches, such as a hospital information system, an electronic medical record, a laboratory information system, a data warehouse, a clinical device information system, or the like, but is not limited thereto, and may receive the electrocardiogram signal data in various methods. Here, the electrocardiogram signal data may be an electrocardiogram signal classified as normal or abnormal by experts having a certain authority and a result of determining whether the electrocardiogram signal is an abnormal signal. In an embodiment, the data receiving unit 211 may receive raw data of an electrocardiogram signal measured from an electrocardiogram signal measuring device or Gini indices calculated from the electrocardiogram signal to input the same to a model. As another embodiment, the data receiving unit 211 may receive a Gini index calculated from an electrocardiogram signal processing apparatus to input the same to a model.

The data learning unit 212 generates an electrocardiogram signal determination model based on the received electrocardiogram signal data. In addition, the data learning unit 212 may perform an operation of calculating Gini indices from raw data of an electrocardiogram signal. The data learning unit 212 may generate an electrocardiogram signal determination model trained based on machine learning. Here, the electrocardiogram signal determination model may be a binary classification model that receives an electrocardiogram signal and classifies the electrocardiogram signal as normal or abnormal, but is not limited thereto. The electrocardiogram signal determination model may be a machine learning model using a perceptron, a support vector machine, a K-nearest neighbor, a decision tree, a random forest algorithm, or a combination thereof, but is not limited thereto, and may use various machine learning methods.

The data learning unit 212 may generate an electrocardiogram signal determination model based on the received electrocardiogram signal data, determine whether an electrocardiogram signal is an abnormal signal by using the electrocardiogram signal determination model, and compare and evaluate a determination result with the input electrocardiogram signal data to generate evaluation data. The data learning unit 212 may update the electrocardiogram signal determination model by receiving the evaluation data as feedback.

The data learning unit 212 may determine a threshold value of a Gini index for determining whether an abnormal signal is present, based on the electrocardiogram signal data.

The data determining unit 213 determines whether the received electrocardiogram signal data is normal or abnormal by using the electrocardiogram signal determination model. Data including a determination result of the data determining unit 213 may be transmitted to the data learning unit 212 to be used as a feedback signal of the electrocardiogram signal determination model.

The processor 220 is configured to generally control the server 200. The processor 220 may include at least one CPU.

The communication unit 230 may be configured to receive an electrocardiogram signal from electrocardiogram signal measuring devices. In addition, the communication unit 230 may be configured to transmit and receive electrocardiogram signal data to/from a system necessary for medical services and researches, such as a hospital information system, an electronic medical record, a laboratory information system, a data warehouse, a clinical device information system, or the like. Also, the communication unit 230 may be configured to receive a determination result of whether an electrocardiogram signal is an abnormal signal and a Gini index from the electrocardiogram signal measuring devices and transmit a threshold value of the Gini index.

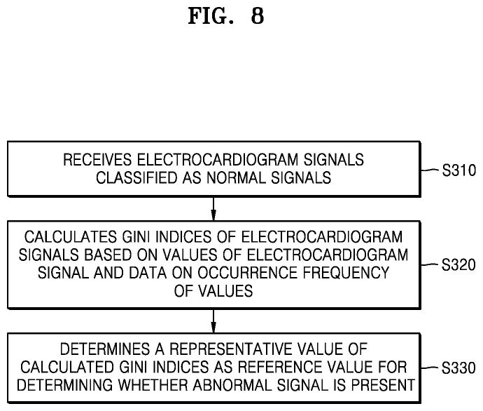

FIG. 8 is a flowchart illustrating a method of determining a reference value of a Gini index.

Referring to FIG. 8, in operation S310, a server receives electrocardiogram signals classified as normal signals.

In operation S320, the server may calculate Gini indices of the electrocardiogram signals which are classified as normal signals. That is, the server may extract values satisfying a determined standard from the received electrocardiogram signals, convert the extracted values into a two-dimensional graph indicating frequencies for a plurality of class sections, rearrange the two-dimensional graph according to an order of magnitude of the frequencies to generate a cumulative graph in which each frequency is converted into a relative frequency, and calculate a Gini index of each electrocardiogram signal based on the cumulative graph.

In operation S330, a representative value of the Gini indices of the electrocardiogram signals which are classified as normal signals may be determined as a threshold value for determining whether an abnormal signal is present. Here, the representative value may be an average value, a median value, a mode value, or an expected value of the electrocardiogram signals which are classified as normal signals, but is not limited thereto.

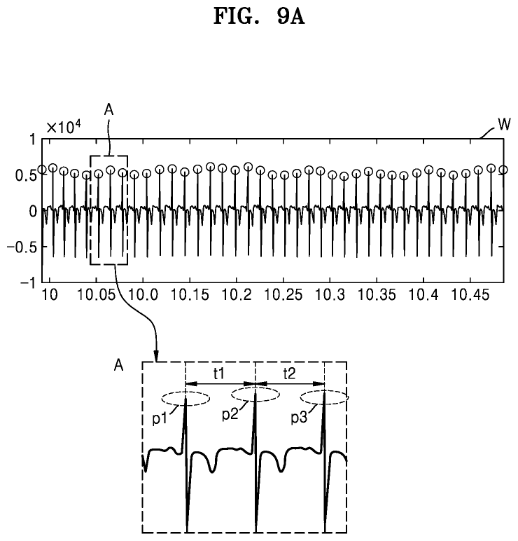

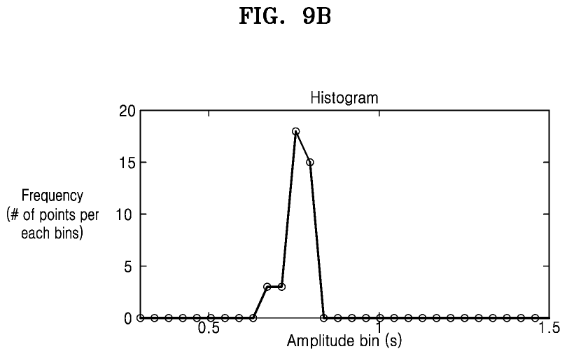

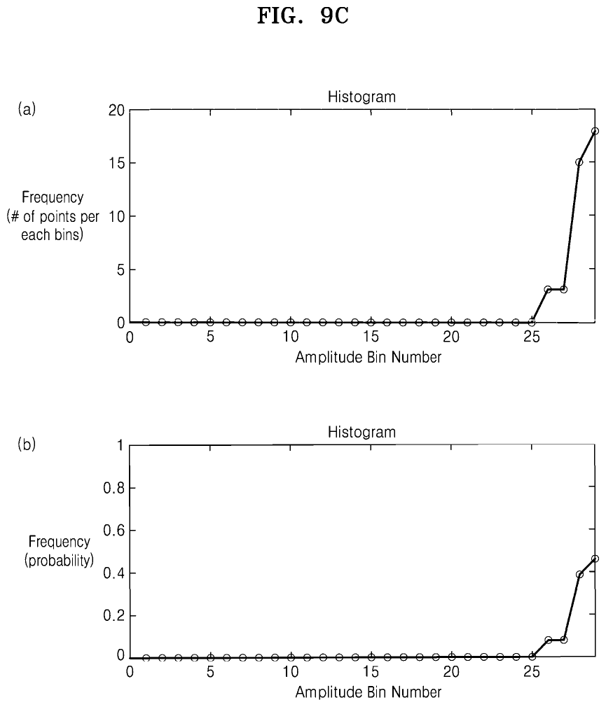

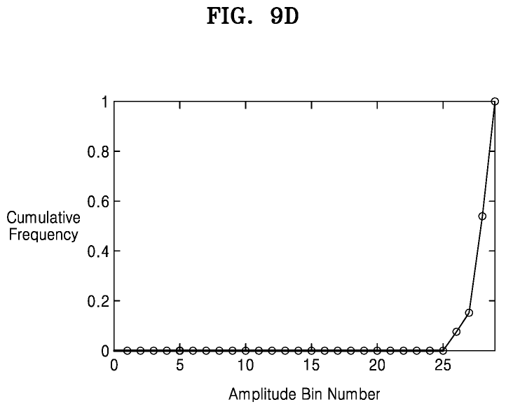

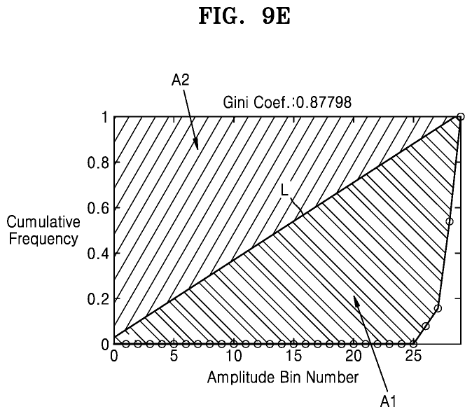

FIG. 9A is an exemplary diagram of a graph of a potential difference with time generated according to an embodiment.

Referring to FIG. 9A, in the present embodiment, an electrocardiogram signal processing apparatus may measure and record a potential difference (μV) between electrodes according to time in minutes. The electrocardiogram signal processing apparatus may divide and calculate an electrocardiogram signal in units of windows of a certain size. In the present embodiment, one window may be set as an area having, for example, continuous 39 R peaks, but is not limited thereto.

An electrocardiogram signal during one heartbeat cycle may include a P wave representing the depolarization of an atrium during the diastole of the ventricles, a QRS-complex representing a state of depolarization of the ventricles, and a T wave representing the normal repolarization of the ventricles.

In the present embodiment, an amplitude of a wave and the R peak of the QRS-complex having a large kurtosis may be set as a reference point of a heartbeat cycle.

The electrocardiogram signal processing apparatus may extract time interval values between reference points with respect to an electrocardiogram signal within a window W. Area A is an enlarged portion of the window W, and includes a first threshold point p1, a second threshold point p2, and a third threshold point p3. Here, a time interval value t1 between the first threshold point p1 and the second threshold point p2 and a time interval value t2 between the second threshold point p2 and the third threshold point p3 may correspond to time interval values between threshold points.

FIG. 9B is an exemplary diagram of a two-dimensional graph generated according to an embodiment. Referring to FIG. 9B, the electrocardiogram signal processing apparatus may convert extract values into a two-dimensional graph.

First, the electrocardiogram signal processing apparatus sets a class section. As an embodiment, a class section may be set by dividing a time interval which may sufficiently include one cycle of a normal heartbeat according to a certain standard. In the present embodiment, each class section may be set by diving 1.5 seconds, which is one period, into 30 sections, which is a determined number.

The electrocardiogram signal processing apparatus distributes the extracted values to each corresponding class section and calculates the number of frequencies of each class section to display the number of frequencies in a two-dimensional graph. Here, in the two-dimensional graph, a frequency of a class section may be represented by one axis (e.g., y-axis), and a class value may be represented by the other axis (e.g., x-axis).

FIG. 9C is an exemplary diagram of a graph rearranged in the order of magnitude of frequencies generated according to an embodiment. Referring to FIG. 9C, the electrocardiogram signal processing apparatus may rearrange an order of the class sections in the two-dimensional graph according to an order of magnitude of the frequencies.

In step (a), the magnitude of the frequencies may be sequentially arranged in an ascending order, a new index may be assigned to each class section, and the assigned index may be used as one axis to generate a rearranged two-dimensional graph.

In step (b), the rearranged two-dimensional graph may be converted into a two-dimensional graph expressed as a relative frequency obtained by dividing frequency values of class sections by a total frequency value. Accordingly, an influence of setting the number of heartbeat cycles included in the window W may be minimized.

FIG. 9D is an exemplary diagram of a cumulative graph for frequencies generated according to an embodiment. Referring to FIG. 9D, the electrocardiogram signal processing apparatus may convert the two-dimensional graph which is converted into the relative frequency into a cumulative graph. Here, the cumulative graph may be a Lorentz curve having a maximum value of 1.

FIG. 9E is a diagram illustrating an operation of calculating a Gini index according to an embodiment. Referring to FIG. 9E, the electrocardiogram signal processing apparatus may set a reference line L connecting points where a cumulative value of the relative frequency becomes 1 from an origin of the cumulative graph, and calculate a Gini index, which is a coefficient representing a ratio of a first area Al between the reference line L and the cumulative graph, and a second area A2 at an upper end of the reference line L.

According to the present embodiment, the Gini index calculated by using the R peak as a reference point may be expressed by the following equation.

$$RRIG = \frac{{\sum }_{i = 1}^{N} {\sum }_{j = 1}^{N} \vert {p}_{i} - {p}_{j} \vert}{2 N {\sum }_{i = 1}^{N} {p}_{i}}$$

Here, i and j are indices assigned to rearranged class sections, and are relative frequencies of each class section, and N is the total number of class sections.

An electrocardiogram signal according to an embodiment of the present disclosure may determine whether an electrocardiogram signal is an abnormal signal with higher accuracy than a method in the related art in a situation where there is a regular fluctuation other than atrial fibrillation.

The technical effects of the present disclosure are not limited to those mentioned above, and other technical effects not mentioned will be clearly understood by the one of ordinary in the art from the following description.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An electrocardiogram signal processing method comprising:
receiving, with an electrocardiogram signal processing apparatus, an electrocardiogram signal;
extracting, with the electrocardiogram signal processing apparatus, values satisfying a determined standard from the electrocardiogram signal and converting extracted values into a two-dimensional graph representing frequencies of a plurality of class sections;
generating, the electrocardiogram signal processing apparatus, a cumulative graph in which an order of the plurality of class sections is rearranged in an order of magnitude of the frequencies;
calculating, with the electrocardiogram signal processing apparatus, a Gini index based on the cumulative graph; and
determining, with the electrocardiogram signal processing apparatus, that the electrocardiogram signal is an abnormal signal by using the Gini index.

2. The method of claim 1, wherein determining that the electrocardiogram signal is the abnormal signal further comprises determining that the electrocardiogram signal is the abnormal signal when the Gini index is equal to or less than a preset reference value.

3. The method of claim 2, further comprising determining the preset reference value by analyzing a plurality of electrocardiogram signals obtained from a plurality of users.

4. The method of claim 1, wherein determining that the electrocardiogram signal is the abnormal signal further comprises determining whether the electrocardiogram signal detected from a heart of a user indicates an arrhythmia or atrial fibrillation.

5. The method of claim 1, wherein converting into the two-dimensional graph further comprises obtaining the two-dimensional graph by dividing the plurality of class sections with respect to values of the electrocardiogram signal or time intervals.

6. The method of claim 5, wherein dividing the class section further comprises dividing the plurality of class sections based on a standard of dividing the plurality of class sections that is changeable.

7. The method of claim 1, wherein extracting the values satisfying the determined standard further comprises extracting time values between reference points of a heartbeat cycle, and
converting into the two-dimensional graph further comprises obtaining the two-dimensional graph by dividing the plurality of class sections with respect to time, the two-dimensional graph representing a time interval between the reference points of the heartbeat cycle as frequencies for the plurality of class sections.

8. An electrocardiogram signal processing apparatus comprising:
a controller; and
a communication unit transmitting an electrocardiogram signal;
wherein the controller is configured to:
extract points satisfying a determined standard from the electrocardiogram signal and convert extracted points into a two-dimensional graph expressed as frequencies for a plurality of class sections;
generate a cumulative graph in which an order of the plurality of class sections is rearranged in an order of magnitude of the frequencies; and
calculate a Gini index based on the cumulative graph and determine whether the electrocardiogram signal is an abnormal signal by using the Gini index.

9. The electrocardiogram signal processing apparatus of claim 8, wherein:
the controller is further configured to determine that the electrocardiogram signal is the abnormal signal when the Gini index is equal to or less than a preset reference value.

10. The electrocardiogram signal processing apparatus of claim 9, wherein:
the controller is further configured to determine the reference value by learning through a plurality of electrocardiogram signals obtained from a plurality of users.

11. The electrocardiogram signal processing apparatus of claim 8, wherein
a determination result that the electrocardiogram signal is the abnormal signal comprises information indicative of whether a heart of a user of the electrocardiogram signal develops an arrhythmia or atrial fibrillation.

12. The electrocardiogram signal processing apparatus of claim 8, wherein the controller is further configured to obtain the two-dimensional graph by dividing the plurality of class sections with respect to values of the electrocardiogram signal or time intervals.

13. The electrocardiogram signal processing apparatus of claim 12, wherein the controller is further configured to divide the plurality of class sections based on a standard of dividing the plurality of class sections that is changeable.

14. The electrocardiogram signal processing apparatus of claim 8, wherein: the points satisfying the determined standard are reference points of a heartbeat cycle, and
the controller is further configured to obtain the two-dimensional graph by diving the plurality of class sections with respect to a time interval,
the two-dimensional graph representing a time interval between the reference points of the heartbeat cycle as frequencies for the plurality of class sections.
